(19) United States
(12) Patent Application Publication
(10) Pub. No.: US 2019/0091204 A1
(43) Pub. Date: Mar. 28, 2019

(54) COMPOSITIONS OF DEFERASIROX

(71) Applicant: Lupin Limited, Mumbai (IN)

(72) Inventors: Ketan Bhasale, Pune (IN); Romesh Jha, Pune (IN); Subhasis Das, Pune (IN); Vijaya Kumar Thommandru, Pune (IN)

(21) Appl. No.: 16/085,714

(22) PCT Filed: Mar. 17, 2017
(86) PCT No.: PCT/IB2017/051549

§ 371 Date: Sep. 17, 2018

(30) Foreign Application Priority Data
Mar. 17, 2016 (IN) 201621009418

(51) Int. Cl. A61K 31/4196; A61K 9/20

(52) U.S. Cl. CPC A61K 31/4196; A61K 9/2031; A61K 9/2027; A61K 9/2054

(57) ABSTRACT

The present invention relates to solid oral pharmaceutical compositions comprising Deferasirox or a pharmaceutical acceptable salt thereof and one or more pharmaceutically acceptable excipients and process for preparation thereof.

### FIELD OF THE INVENTION

The present invention relates to solid oral pharmaceutical compositions comprising Deferasirox or a pharmaceutical acceptable salt thereof and one or more pharmaceutically acceptable excipients and process for preparation thereof.

### BACKGROUND OF INVENTION

Deferasirox or 4-[3, 5-bis (2-hydroxyphenyl)-1H-1, 2, 4-triazol-1-yl] benzoic acid is an orally active chelator that is selective for iron (as Fe3+). It is a tridentate ligand that binds iron with high affinity in a 2:1 ratio. Deferasirox is indicated for the treatment of chronic iron overload due to blood transfusions (transfusional hemosiderosis) in patients 2 years of age and older. It is also used for the treatment of chronic iron overload in patients 10 years of age and older with non-transfusion-dependent thalassemia (NTDT) syndromes and with a liver iron concentration (LIC) of at least 5 milligrams of iron per gram of liver dry weight (mg Fe/g dw) and a serum ferritin greater than 300 mcg/L.

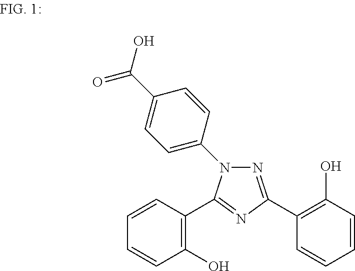 [SMILES: O=C(O)c1ccc(-n2nc(-c3ccccc3O)nc2-c2ccccc2O)cc1]

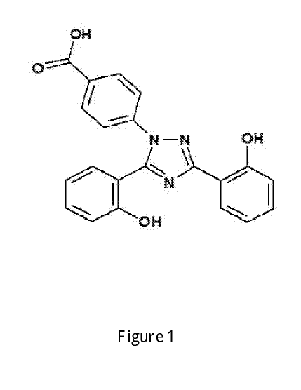

Currently, Deferasirox is approved in the form of tablets, under the brand name JADENU™ 360 mg, 180 mg, 90 mg and in the form of tablets, for oral suspension, under the brand name EXJADE™ 125 mg, 250 mg, 500 mg which is marketed by a Novartis. Deferasirox as depicted in FIG. 1, its process of manufacture and its uses are described in U.S. Pat. No. 6,465,504 B1.

U.S. Pat. Publication No. 20060110446 A1 discloses a dispersible tablet composition of Deferasirox, wherein Deferasirox is present in an amount of from 5 to 40% in weight by weight of the total tablet.

U.S. Pat. Publication No. 20150017241 A1 discloses pharmaceutical compositions comprising Deferasirox and one or more pharmaceutically acceptable excipients, wherein the Deferasirox is present in an amount of from 45% to 60% by weight based on the total weight of the tablet, wherein the composition having reduced release under gastric condition and fast release at near neutral pH or at neutral pH. This patent application discloses composition using poloxamer 188 which is compatible with Deferasirox at physiological pH environment. However, there is a continuing need for new solid forms of Deferasirox and new methods of preparation.

### OBJECTIVES OF INVENTION

The main object of the invention is to provide solid oral pharmaceutical compositions of Deferasirox or a pharmaceutical acceptable salt thereof and a process for preparation thereof. The pharmaceutical compositions of the invention are preferably compositions in the form of tablet.

In another object of the invention is a solid oral pharmaceutical composition comprising Deferasirox or a pharmaceutically acceptable salt thereof and one or more pharmaceutically acceptable excipients, wherein the Deferasirox or a pharmaceutically acceptable salt thereof is present in an amount less than 45% by weight based on the total weight of the composition, wherein the composition having reduced release under gastric condition and fast release at neutral pH or at neutral pH.

In another object of the invention is a solid oral pharmaceutical composition comprising Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition, wherein the composition having reduced release under gastric condition and fast release at neutral pH or at neutral pH.

In another object of the invention is a solid oral pharmaceutical composition having a disintegration time of 5-10 minutes when measured by a standard USP disintegration test.

In another object of the invention is a solid oral pharmaceutical composition comprising Deferasirox or a pharmaceutically acceptable salt thereof and one or more pharmaceutically acceptable excipients, wherein the composition is free of surfactant, wherein the composition having reduced release under gastric condition and fast release at neutral pH or at neutral pH.

In another object of the invention is a solid oral pharmaceutical composition which is further comprising enteric coating.

In another object of the invention is a solid pharmaceutical composition which is bioequivalent to the marketed composition of 360 mg of Deferasirox tablets.

### DETAILED DESCRIPTION OF THE INVENTION

The present invention relates to solid oral pharmaceutical compositions of Deferasirox or a pharmaceutical acceptable salt thereof and a process for preparation thereof. More particulalry, pharmaceutical compositions are in the form of tablet and process for preparation thereof.

Solid oral pharamaceutical compositions of invention have comparable in-vitro dissolution profile with marketed tablet formulation of Deferasirox. More preferably, pharmaceutical compositions of invention are bioequivalent to marketed tablet formulation of Deferasirox.

“Deferasirox” as depicted in FIG. 1 used in the present invention is in the form of base or pharmaceutically acceptable derivative like esters(s) or salt(s) or enantiomer(s) or polymorph(s) or solvates thereof. Preferably Deferasirox is in the form of base.

Solid oral pharmaceutical compositions of Deferasirox or a pharmaceutically acceptable salt thereof according to the invention comprise but are not limited to powders, tablets (single layered tablets, multilayered tablets, mini tablets, bioadhesive tablets, caplets, matrix tablets, tablet within a tablet, mucoadhesive tablets, modified release tablets, pulsatile release tablets, and timed release tablets), pellets, beads, granules, sustained release formulations, capsules, microcapsules, tablets in capsules, microspheres, matrix formulations, microencapsulation.

In one embodiment, a solid oral pharmaceutical composition in the form tablet comprising Deferasirox or a pharmaceutically acceptable salt thereof and one or more pharmaceutically acceptable excipients, wherein the Deferasirox or a pharmaceutically acceptable salt thereof is present in an amount less than 45% by weight based on the total weight of the composition, wherein the composition having reduced release under gastric condition and fast release at neutral pH or at neutral pH.

The term ‘pharmaceutically acceptable excipients’ used in the pharmaceutical compositions of invention comprise but not limited to diluents, binders, pH stabilizing agents, disintegrants, surfactants, glidants, lubricants, suspending agents, flavouring agents, sweetening agents, buffers or preservatives.

The amount of excipient(s) employed will depend upon how much active agent is to be used. One excipient(s) can perform more than one function.

Binders as used in the invention comprises but are not limited to, starches such as potato starch, wheat starch, corn starch; microcrystalline cellulose such as products known under the registered trademarks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents as used in the invention comprises but not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants as used in the invention comprises but not limited to magnesium stearate, polyethylene glycol, glyceryl behenate, mineral oil, sodium stearyl fumarate, stearic acid, hydrogenated vegetable oil and talc.

Glidants comprises but not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Disintegrants comprises but not limited to starches; clays; celluloses; alginates; gums; cross-linked polymers, e.g., cross-linked polyvinyl pyrrolidone or crospovidone, e.g., POLYPLASDONE XL, cross-linked sodium carboxymethylcellulose or croscarmellose sodium, e.g., AC-DI-SOL from FMC; and cross-linked calcium carboxymethylcellulose; soy polysaccharides; and guar gum. Use of disintegrant according to the invention facilitates in the release of drug in the latter stage and thereby completely releasing the drug from the dosage form.

Examples of sweetening agents and flavouring agents comprises but not limited to sugar alcohols, sugars, liquid glucose, sucrose, sachharine sodium, banana flavouring, vanilla flavouring, tutti frutty flavor, xylitol, sorbitol, mannitol, erythritol and the like.

Examples of sugar alcohols that may be used in the present invention include but not limited to sorbitol, erythritol, D-mannitol, sucrose and the like, wherein the most preferable sugar alcohol is sorbitol.

Examples of preservatives used in the invention comprises but are not limited to, sodium benzoate, chlorhexidine; methyl paraben; propyl paraben; butyl paraben and their salts; diazolidinyl urea; quaternary compounds like benzalkonium chloride and cetylpyridinium chloride, phenyl ethyl alcohol and the like. More preferably, compositions of the invention comprises sodium benzoate as preservative.

Examples of surfactants include, but are not limited to, poloxamers, heptadecaethylene oxycetanol, lecithins, sorbitol monooleate, polyoxyethylene sorbitol monooleate, polyoxyethylene stearate, polyoxyethylen sorbitan monolaurate, polysorbates for example 20, 40, 60 or 80, sorbitan mono-palmitate, sodium salts of fatty alcohol-sulaftes such as sodium lauryl sulfate, sodium dodecylsulfate, sodium salts of sulfosuccinates such as sodium dioctylsulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, -monostearate or -monooleate, ethers of fatty alcohols with polyhydroxyethylene, esters of fatty acids with polyhydroxyethylene, copolymers of ethylenoxide and propylenoxide (Pluronic®) and ethoxylated triglycerides or tyloxapol.

It is to be understood for the purpose of invention, the dose of Deferasirox or its pharmaceutically acceptable salt form is between 1 mg to 800 mg. More preferably the dose is between 50 mg to 400 mg. Most preferably the dose is selected from 90 mg, 180 mg or 360 mg.

In one embodiment, a solid oral pharmaceutical composition in the form tablet comprising 90 mg Deferasirox or a pharmaceutically acceptable salt thereof and one or more pharmaceutically acceptable excipients, wherein the Deferasirox or a pharmaceutically acceptable salt thereof is present in an amount less than 45% by weight based on the total weight of the composition, wherein the composition having reduced release under gastric condition and fast release at neutral pH or at neutral pH.

In one embodiment, a solid oral pharmaceutical composition in the form tablet comprising 180 mg Deferasirox or a pharmaceutically acceptable salt thereof and one or more pharmaceutically acceptable excipients, wherein the Deferasirox or a pharmaceutically acceptable salt thereof is present in an amount less than 45% by weight based on the total weight of the composition, wherein the composition having reduced release under gastric condition and fast release at neutral pH or at neutral pH.

In one embodiment, a solid oral pharmaceutical composition in the form tablet comprising 360 mg Deferasirox or a pharmaceutically acceptable salt thereof and one or more pharmaceutically acceptable excipients, wherein the Deferasirox or a pharmaceutically acceptable salt thereof is present in an amount less than 45% by weight based on the total weight of the composition, wherein the composition having reduced release under gastric condition and fast release at neutral pH or at neutral pH.

In another embodiment, a solid oral pharmaceutical composition in the form of tablet comprising 90 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition, wherein the composition having reduced release under gastric condition and fast release at neutral pH or at neutral pH.

In another embodiment, a solid oral pharmaceutical composition in the form of tablet comprising 180 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition, wherein the composition having reduced release under gastric condition and fast release at neutral pH or at neutral pH.

In another embodiment, a solid oral pharmaceutical composition in the form of tablet comprising 360 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition, wherein the composition having reduced release under gastric condition and fast release at neutral pH or at neutral pH.

In another embodiment, a solid oral pharmaceutical composition of invention is capsule.

In another embodiment, a solid oral pharmaceutical composition in the form of tablet comprising 360 mg Deferasirox or a pharmaceutically acceptable salt thereof and one or more pharmaceutically acceptable excipients, wherein the Deferasirox or a pharmaceutically acceptable salt thereof is present in an amount less than 45% by weight based on the total weight of the composition having a disintegration time of 2-7 minutes when measured by a standard USP disintegration test.

In another embodiment, a solid oral pharmaceutical composition in the form of tablet comprising 360 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition having a disintegration time of 5-10 minutes when measured by a standard USP disintegration test.

In another embodiment, a solid oral pharmaceutical composition in the form of tablet comprising 360 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition such that when the composition is tested in-vitro by USP Apparatus II (Paddle) method of U.S. Pharmacopoeia at 50 rpm, at 37° C. in pH 6.8 phosphate buffer with 0.25% Tween, the release rate of at least 70% by weight within 15 minutes.

In another embodiment, a solid oral pharmaceutical composition in the form of tablet comprising 360 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition such that when the composition is tested in-vitro by USP Apparatus II (Paddle) method of U.S. Pharmacopoeia at 50 rpm, at 37° C. in pH 6.8 phosphate buffer with 0.25% Tween, the release rate of at least 80% by weight within 30 minutes.

In another embodiment, a solid oral pharmaceutical composition in the form of tablet comprising 360 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition such that when the composition is tested in-vitro by USP Apparatus II (Paddle) method of U.S. Pharmacopoeia at 50 rpm, at 37° C. in pH 6.8 phosphate buffer with 0.25% Tween, the release rate of at least 80% by weight within 45 minutes.

In another embodiment, a solid oral pharmaceutical composition in the form of tablet comprising Deferasirox or a pharmaceutically acceptable salt thereof and one or more pharmaceutically acceptable excipients, wherein the composition is free of one or more surfactants selected from sodium lauryl sulfate, betain, quaternary ammonium salts, polysorbates, sorbitan esters and a poloxamer, wherein the composition having reduced release under gastric condition and fast release at neutral pH or at neutral pH.

In another embodiment, a solid oral pharmaceutical composition in the form of tablet comprising 360 mg Deferasirox or a pharmaceutically acceptable salt thereof and one or more pharmaceutically acceptable excipients, wherein the composition is free of surfactant, wherein the composition having reduced release under gastric condition and fast release at neutral pH or at neutral pH.

In another embodiment, a solid oral pharmaceutical composition in the form of tablet comprising 180 mg Deferasirox or a pharmaceutically acceptable salt thereof and one or more pharmaceutically acceptable excipients, wherein the composition is free of surfactant, wherein the composition having reduced release under gastric condition and fast release at neutral pH or at neutral pH.

In another embodiment, a solid oral pharmaceutical composition in the form of tablet comprising 90 mg Deferasirox or a pharmaceutically acceptable salt thereof and one or more pharmaceutically acceptable excipients, wherein the composition is free of surfactant, wherein the composition having reduced release under gastric condition and fast release at neutral pH or at neutral pH.

In another embodiment, a solid oral pharmaceutical composition which is further comprising enteric coating.

In another embodiment, a solid oral pharmaceutical composition comprising Deferasirox or a pharmaceutically acceptable salt thereof and one of more pharmaceutically acceptable excipients, which is further comprising enteric coating.

In another embodiment, a solid oral pharmaceutical composition in the form of tablet comprising Deferasirox or a pharmaceutically acceptable salt thereof and one of more pharmaceutically acceptable excipients, which is further comprising enteric coating.

In another embodiment, a solid oral pharmaceutical composition in the form of tablet comprising 360 mg Deferasirox or a pharmaceutically acceptable salt thereof and one of more pharmaceutically acceptable excipients, which is further comprising enteric coating.

In another embodiment, a solid oral pharmaceutical composition in the form of tablet comprising 180 mg Deferasirox or a pharmaceutically acceptable salt thereof and one of more pharmaceutically acceptable excipients, which is further comprising enteric coating.

In another embodiment, a solid oral pharmaceutical composition in the form of tablet comprising 90 mg Deferasirox or a pharmaceutically acceptable salt thereof and one of more pharmaceutically acceptable excipients, which is further comprising enteric coating.

In another embodiment, a solid oral pharmaceutical composition comprising Deferasirox or a pharmaceutically acceptable salt thereof and one of more pharmaceutically acceptable excipients which is bioequivalent to the marketed composition of 360 mg of Deferasirox tablets.

In another embodiment, a solid oral pharmaceutical composition in the form of tablet comprising 360 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition wherein the composition is bioequivalent to the marketed composition of 360 mg of Deferasirox tablets.

In another embodiment, a process of preparing solid oral pharmaceutical compositions of invention.

In another embodiment, a solid oral pharmaceutical compositions of invention can be used for treating diseases which cause an excess of metal in human or animal body or are causes by excess of metal in a human or animal body.

The following examples are illustrative of the present invention, and the examples should not be considered as limiting the scope of this invention in any way, as these examples and other equivalents thereof will become apparent to those versed in the art, in the light of the present disclosure, and the accompanying claims.

### EXAMPLES

### Example 1

| S. No. | Ingredients | % w/w |
|---|---|---|
| 1 | Deferasirox (360 mg) | 65.45 |
| 2 | Microcrystalline | 19.68 |
| | Cellulose | |
| 3 | Povidone | 4.00 |
| 4 | Crospovidone | 8.82 |
| 7 | Colloidal anhydrous | 0.55 |
| | silica | |
| 8 | Magnesium Stearate | 1.50 |
| 9 | Film Coating | 3.00 |
| 10 | Water | q.s. |

Procedure:
1. Deferasirox, microcrystalline cellulose and crospovidone sifted together.
2. Dissolve Povidone in water to prepare the solution.
3. Prepare granules of the Deferasirox mixture of step 1 using solution of step 2.
4. Dry the granules and sift through the sieve.
5. Mix and blend the granules with crospovidone, colloidal anhydrous silica.
6. Lubricate the blend of with magnesium stearate.
7. Compress the blend of step 6 into tablet.
8. Film coat the tablet of step 7.

### Example 2

| S. No. | Ingredients (Intragranular) | % w/w |
|---|---|---|
| 1 | Deferasirox (360 mg) | 40.00 |
| 2 | Microcrystalline Cellulose | 12.84 |
| 3 | Lactose Monohydrate | 0.30 |
| 4 | Povidone | 2.44 |
| 5 | Poloxamer | 0.14 |
| 6 | Crospovidone | 5.39 |
| 7 | Magnesium Stearate | 0.92 |
| 8 | Film Coating | 3.00 |
| 9 | Water | q.s. |

Procedure:
1. Lactose, microcrystalline cellulose and crospovidone sifted together.
2. Dissolve Povidone and Poloxamer in water to prepare the solution.
3. Add Deferasirox and stir to prepare dispersion.
4. Spray the dispersion of step 3 on sifted material of step 1 in fluid bed processor.
5. Dry the granules and sift through the sieve.
6. Mix and blend the granules with crospovidone, colloidal anhydrous silica.
7. Lubricate the blend of with magnesium stearate.
8. Compress the blend of step 7 into tablet.
9. Film coat the tablet of step 8.

In-Vitro Dissolution Study:

Table 1 given below shows the comparative dissolution profile of Deferasirox tablet of Example 1 of the present invention (Test) & JADENU® 360 mg tablets (Reference) carried out in-vitro by USP Apparatus II (Paddle) method of U.S. Pharmacopoeia at 50 rpm, at 37° C. in pH 6.8 pH 6.8 phosphate buffer with 0.25% Tween. The release profile (cumulative % of drug released) is given in Table 1.

TABLE 1

Cumulative % Drug Release

| Time points | JADENU ® (Deferasirox | |
|---|---|---|
| (min) | Tablets 360 mg) | Example |
| 10 | 62 | 56 |
| 15 | 71 | 79 |
| 30 | 80 | 93 |
| 45 | 83 | 95 |

1. A solid oral pharmaceutical composition comprising Deferasirox or a pharmaceutically acceptable salt thereof and one or more pharmaceutically acceptable excipients, wherein the deferasirox or a pharmaceutically acceptable salt thereof is present in an amount that is less than 45% by weight based on the total weight of the composition, said composition having reduced release under gastric condition and fast release at near neutral pH or at neutral pH.

2. The solid oral pharmaceutical composition according to claim 1, in tablet form, pellet form or multi-particulate form.

3. The solid oral pharmaceutical composition according to claim 1, in tablet form possessing a disintegration time of about 2-7 minutes when measured by a standard USP disintegration test.

4. The solid oral pharmaceutical composition according to claim 1, in tablet form comprising 360 mg of Deferasirox or a pharmaceutically acceptable salt thereof.

5. A solid oral pharmaceutical composition comprising Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition, wherein the composition having reduced release under gastric condition and fast release at neutral pH or at neutral pH.

6. The solid oral pharmaceutical composition according to claim 5, in tablet form, pellet form or multi-particulate form.

7. The solid oral pharmaceutical composition according to claim 5, in tablet form possessing a disintegration time of about 5-10 minutes when measured by a standard USP disintegration test.

8. The solid oral pharmaceutical composition according to claim 5, in tablet form comprising 360 mg of Deferasirox or a pharmaceutically acceptable salt thereof.

9. A solid oral pharmaceutical composition in the form of tablet comprising 360 mg Deferasirox or a pharmaceutically acceptable salt thereof and one or more pharmaceutically acceptable excipients, wherein the Deferasirox or a pharmaceutically acceptable salt thereof is present in an amount more than 60% by weight based on the total weight of the composition such that when the composition is tested in-vitro by USP Apparatus II (Paddle) method of U.S. Pharmacopoeia at 50 rpm, at 37° C. in pH 6.8 phosphate buffer with 0.25% Tween, the release rate of at least 70% by weight within 15 minutes.

10. The solid oral pharmaceutical composition according to claim 9, used for treating diseases which cause an excess of metal in human or animal body or are causes by excess of metal in a human or animal body.
